(19) United States
(12) Patent Application Publication
(10) Pub. No.: US 2023/0323265 A1
(43) Pub. Date: Oct. 12, 2023

(54) BIOREACTOR SYSTEM

(71) Applicant: CORNING INCORPORATED, Corning, NY (US)

(72) Inventors: Gregory Roger Martin, Acton, ME (US); Allison Jean Tanner, Portsmouth, NH (US)

(21) Appl. No.: 18/023,227

(22) PCT Filed: Aug. 26, 2021
(86) PCT No.: PCT/US2021/047658

§ 371 Date: Feb. 24, 2023

Related U.S. Application Data
(60) Provisional application No. 63/072,517, filed on Aug. 31, 2020.

(51) Int. Cl. C12M 1/00; C12M 3/00

(52) U.S. Cl. CPC C12M 23/50; C12M 23/44; C12M 29/04; C12M 23/48; C12M 29/20; C12M 41/14

(57) ABSTRACT

A cell culture system is provided that includes at least one multi-layered vessel for culturing cells, and a cabinet comprising an interior cavity enclosed by one or more sidewalls. The cabinet being configured to house the multi-layered vessel within the interior cavity. The multi-layered vessel includes a cell culture space within the multi-layered vessel. The cabinet can change an orientation of the multi-layered vessel from an upright orientation to a tilted orientation.

### CROSS-REFERENCE TO RELATED APPLICATIONS

This application claims the benefit of priority under 35 U.S.C. § 119 of U.S. Provisional Application Ser. No. 63/072,517 filed on Aug. 31, 2020, the content of which is relied upon and incorporated herein by reference in its entirety.

### FIELD

The present disclosure relates to systems and methods for culturing cells and, in particular, systems and methods that that combine multiple cell culture vessels to enable high yields of cell culture products while being minimizing space requirements and manual handling of the vessels.

### BACKGROUND

Many types of cell culture articles are constructed to provide stacked or stackable units for culturing cells. For example, T-flasks are typically made to have flat top and bottom surfaces that allow T-flasks to be stacked, providing space savings. Some modified T-flasks have multiple parallel culture surfaces within the flask to reduce time and effort associated with filling and emptying. Other culture apparatuses are multi-component assemblies having a plurality of parallel or stacked culture surfaces. With most of such stacked culture assemblies, each culture layer is isolated to reduce hydrostatic pressure on the lower culture layers. As the number of stacked layers increases, the potential effect of hydrostatic pressure increases.

One exemplary cell culture article is Coming's HYPERStack™ system. The HYPERStack™ system includes multiple modules formed of individual stackette layers that can be interconnected by flexible tubes that connect to tube connectors. The modules are interconnected for filling and emptying the HYPERStack™ system. Valves and other devices may be used to control fluid flow into and out of the HYPERStack™ system. The HYPERStack™ 36-layer vessel has lowered the barrier of entry for users wanting to conduct phase I and II clinical trials. However, current protocols for using the HYPERStack™ can be labor intensive, requiring manual manipulation. For example, the current processes for filling and emptying the HYPERStack™ system involve tilting the HYPERStack™ system at various stages in order to yield better results. This tilting not only may require the attention and manual manipulation by users, but can also lead to inconsistent results where the protocols are applied inconsistently. Where multiple HYPERStack™ vessels are used for larger cell culture needs, this manual manipulation requires an increasing amount of labor by users during critical timepoints of cell or virus manufacturing, such as during seeding, transfection, refeeding, and harvesting. Each individual that handles the vessels can result in variation within the virus manufacturing process that decreases efficiency. Damage to the vessel can also occur during these manipulation steps.

In addition, as customers need larger cell cultures (e.g., into or past phase III clinical trials), the HYPERStack™ system can scale in a way requires a large footprint and the space requirements can become unmanageable for some users. The use of manual manipulation of the HYPERStack™ system during filling and emptying can exacerbate the space issue, as the use for manual manipulation often results in an inefficient use of space between multiple HYPERStack™ units.

What is needed are systems and methods for cell culture and virus manufacture that are more controlled, require less manual labor, and are space efficient for scaling up manufacturing.

### BRIEF SUMMARY

According to embodiments of this disclosure, a cell culture system is provided that includes at least one multi-layered vessel for culturing cells, the multi-layered vessel including a cell culture space within the multi-layered vessel; and a cabinet having an interior cavity enclosed by one or more sidewalls, the cabinet able to house the multi-layered vessel within the interior cavity. The cabinet can change an orientation of the multi-layered vessel from an upright orientation to a tilted orientation.

As an aspect of some embodiments, the system further includes at least one sensor to sense a property within the cell culture space. The sensor can include at least one of a confluence monitor and an analyte monitor. The sensor can integrated into the multi-layered vessel. In another aspect, the sensor is attached to the cabinet and arranged to sense the property within the cell culture space when the multi-layered vessel is disposed in the cabinet.

In a further aspect of some embodiments, the multi-layered vessel includes at least one sensor window through which the sensor is configured to sense the property within the cell culture space.

In some embodiments, the cabinet comprises includes support surfaces each configured to support the at least one multi-layered vessel multi-layered vessel.

According to aspects of some embodiments, the at least one multi-layered vessel includes a plurality of multi-layer cell culture modules. At least some of the plurality of multi-layer cell culture modules vessels can be coupled to one another.

The multi-layered vessel can include an inlet and an outlet, where the inlet is configured to supply liquid media to the cell culture space and the outlet is configured for passing liquid or gas into or out of the cell culture space. As an aspect of some embodiments, the inlet is disposed in a lower portion of the multi-layered vessel. The outlet can be disposed in an upper portion of the multi-layered vessel.

### BRIEF DESCRIPTION OF THE DRAWINGS

FIG. 1 is a perspective view of a cell culture apparatus, according to one or more embodiments shown and described herein.

FIG. 2 is a diagrammatic of multiple stackette layers for use with the cell culture apparatus of FIG. 1, according to one or more embodiments shown and described herein.

FIG. 3 is a side view of a multi-position support that supports the cell culture apparatus of FIG. 1 in an upright configuration, according to one or more embodiments shown and described herein.

FIG. 4 is a perspective view of the multi-position support of FIG. 3, according to one or more embodiments shown and described herein.

FIG. 5 is a plan view of the multi-position support of FIG. 4, according to one or more embodiments shown and described herein.

FIG. 6 is a side view of the multi-position support of FIG. 3 in a tilted configuration, according to one or more embodiments shown and described herein.

FIG. 7 is an end view of the multi-position support of FIG. 6 in the tilted configuration, according to one or more embodiments shown and described herein.

FIG. 8 is a cross-section view of a 2D cell culture module, according to one or more embodiments shown and described herein.

FIG. 9 is a cross-section view of a 3D cell culture module, according to one or more embodiments shown and described herein.

FIG. 10 is a cell culture vessel having multiple cell culture modules, according to one or more embodiments shown and described herein.

FIG. 11A shows a side view of the cell culture vessel of FIG. 10 during the initial stage of a filling operation, according to one or more embodiments shown and described herein.

FIG. 11B shows a side view of the cell culture vessel of FIG. 11A half-way through the filling operation, according to one or more embodiments shown and described herein.

FIG. 11C shows a side view of the cell culture vessel of FIGS. 11A and 11B upon completion of the filling operation, according to one or more embodiments shown and described herein.

FIG. 12 is a side view of a cell culture system including a cabinet housing multiple cell culture vessels, according to one or more embodiments shown and described herein.

FIG. 13 is a side view of a cell culture system including a cabinet and gas-impermeable enclosure, according to one or more embodiments shown and described herein.

FIG. 14A is a side schematic view of a cell culture system in an upright configuration on a cart, according to one or more embodiments shown and described herein.

FIG. 14B is a side schematic view of a cell culture system in a tilted configuration on a cart, according to one or more embodiments shown and described herein.

The drawings are not necessarily to scale. Like numbers used in the figures refer to like components, steps and the like. However, it will be understood that the use of a number to refer to a component in a given figure is not intended to limit the component in another figure labeled with the same number. In addition, the use of different numbers to refer to components is not intended to indicate that the different numbered components cannot be the same or similar.

### DETAILED DESCRIPTION

In the following detailed description, reference is made to the accompanying drawings that form a part hereof, and in which are shown by way of illustration several specific embodiments of devices, systems and methods. It is to be understood that other embodiments are contemplated and may be made without departing from the scope or spirit of the present disclosure. The following detailed description, therefore, is not to be taken in a limiting sense.

All scientific and technical terms used herein have meanings commonly used in the art unless otherwise specified. The definitions provided herein are to facilitate understanding of certain terms used frequently herein and are not meant to limit the scope of the present disclosure.

As used in this specification and the appended claims, the singular forms “a,” “an,” and “the” encompass embodiments having plural referents, unless the content clearly dictates otherwise. As used in this specification and the appended claims, the term “or” is generally employed in its sense including “and/or” unless the content clearly dictates otherwise.

As used herein, “have,” “having,” “include,” “including,” “comprise,” “comprising” or the like are used in their open-ended sense, and generally mean “including, but not limited to.”

“About” modifying, for example, the quantity of an ingredient in a composition, concentrations, structural dimensions, volumes, process temperature, process time, yields, flow rates, pressures, viscosities, and like values, and ranges thereof, employed in describing the embodiments of the disclosure, refers to variation in the numerical quantity that can occur, for example: through typical measuring and handling procedures used for preparing materials, compositions, composites, concentrates, or use formulations; through inadvertent error in these procedures; through differences in the manufacture, source, or purity of starting materials or ingredients used to carry out the methods; and like considerations. The term “about” also encompasses amounts that differ due to aging of a composition or formulation with a particular initial concentration or mixture, and amounts that differ due to mixing or processing a composition or formulation with a particular initial concentration or mixture.

In vitro culturing of cells provides material necessary for research in pharmacology, physiology, and toxicology. Recent advances in pharmaceutical screening techniques allow pharmaceutical companies to rapidly screen vast libraries of compounds against therapeutic targets. These large-scale screening techniques require large numbers of cells grown and maintained in vitro. Maintaining these large numbers of cells requires large volumes of cell growth media and reagents, and large numbers and types of laboratory cell culture containers and laboratory equipment. This activity can be labor intensive.

Cell culture vessels have been developed to provide an increased surface area for cell growth while also providing necessary gas exchange. These systems also employ traditional cell culture vessels including common flasks, roller bottles, cell culture dishes, and multi-layered cell growth vessels, including multi-layer flasks, multi-layer cell culture dishes, bioreactors, cell culture bags, and like articles, which may include specialized surfaces designed to enhance the cell culture parameters including growth density and differentiation factors. Examples of closed system cell culture articles that have been specifically developed for high-yield cell growth include the HYPERFlask® and HYPERStack® products (available from Corning, Inc.), which have a gas permeable film that provides the cell growth surfaces and allow gas exchange with the ambient environment.

The present disclosure describes, among other things, systems and methods for cell and cell-derived product production in a more controlled and/or compact manner than previous multi-layered vessels. Described herein are cell culture systems and methods that may provide closed and automated or semi-automated systems that perform culturing of anchorage dependent, or adherent, cells or three-dimensional (“3D”) cell culture using one or more cell culture vessels. The system and methods enable dense cell culture footprints to save space and increase yields in a cell culture production facility.

According to embodiments of this disclosure, the cell culture vessels may include one or more cell culture surfaces and at least one port for allowing materials to flow in and out of the cell culture vessel. In embodiments, the system is configured to automatically fill the one or more cell culture vessels with cell culture medium, to release the cells cultured within the one or more cell culture vessels from the one or more cell culture surfaces, and to empty (e.g., harvest) the cultured cells from the one or more cell culture vessels. In embodiments, the system is a closed system. As used herein, a system being “closed” means that the cell culture vessels can be operated without being open to the outside environment during culturing processes. Embodiments provide high-yield cell culturing systems and methods with minimized spatial requirements (e.g., small footprint) and more controlled manipulation of cell culture vessels to reduce variability in cell culture conditions.

Embodiments of this disclosure include cell culturing systems and methods that include or use one or more cell culture vessels (e.g., at least one cell culture vessel, more than one cell culture vessel, two or more cell culture vessels, etc.) that may be configured to culture a plurality of anchorage dependent, or adherent, cells, or for 3D cell cultures. In embodiments, cell culture vessels include a plurality of parallel cell culture surfaces within a plurality of a stacked or multilayer units, compartments, or modules (e.g., the plurality cell culture surfaces are parallel to one another). Nonetheless, according to some embodiments, nearly any cell culture vessel can be adapted for use with systems described herein. For example, any cell culture vessel having a plurality of stacked layers or that can be stacked to form layers can be adapted to be used by the systems described herein. Examples of such cell culture vessels include T-flasks, TRIPLE-FLASK cell culture vessels (Nunc., Intl.), HYPERFLASK cell culture vessels (Corning, Inc.), CELLSTACK culture chambers (Corning, Inc.), CELLCUBE modules (Corning, Inc.), HYPERSTACK cell culture vessels (Corning, Inc.), CELL FACTORY culture apparatuses (Nunc, Intl.), and cell culture articles/vessels as described in WO 2007/015770, entitled “MULTILAYERED CELL CULTURE APPARATUS,” and published Feb. 8, 2007, which is hereby incorporated by reference in its entirety to the extent that it does not conflict with the present disclosure. Of course, cell culture vessels that do not have stacked layers or that are not generally stackable may be used in some embodiments.

The multi-layer cell culture vessel can include cell culture modules, which include a plurality of growth or culture surfaces in cell culture chambers, coupled together via manifolds to form the cell culture vessel. The cell culture vessel can be further coupled to additional cell culture vessels via manifolds to form stacked or horizontally coupled cell culture devices. In some embodiments, the manifold can include an integral column structure that is formed as a monolithic part of the manifold. The column structure includes an inlet port and provides at least part of a fluid flow pathway from the inlet port that is in fluid communication with the individual cell culture chambers within the cell culture modules. The manifolds and associated column structures may provide a closed system where the column structures can be connected to flexible tubing to isolate the cell culture chambers from the environment during use of the cell culture apparatuses.

The cell culture vessels may include a plurality of cell culture surfaces coupled via a manifold. The plurality of culture surfaces may be stacked in a multi-layer configuration. The manifold may include a plurality of fluidly coupled ports that serve to isolate individual or groups of cell culture chambers. Generally, the cell, or growth, culture surfaces are positioned parallel to the ground surface during cell culture processes. To distribute material such as, e.g., cell culture medium, within the cell culture vessels, the cell culture vessels may be positioned, or moved, such that the plurality of cell culture surfaces are not positioned parallel to the ground surface such that material may be distributed evenly into all of the chambers/units and across all of the plurality of cell culture surfaces.

The cell culture vessels can include multiple cell culture modules, each having cell culture spaces and/or including multi-layered cell growth surfaces. In further embodiments, the cell culture vessels can be grouped together to enable large-scale cell growth and thus large-scale production of virus, extracellular vesicles, cells, and other cell-derived products.

A cell culture vessel, or portions thereof, as described herein may be formed from any suitable material. Preferably, materials intended to contact cells or culture media are compatible with the cells and the media. Typically, cell culture units are formed from polymeric material. Examples of suitable polymeric materials include polystyrene, polymethylmethacrylate, polyvinyl chloride, polycarbonate, polysulfone, polystyrene copolymers, fluoropolymers, polyesters, polyamides, polystyrene butadiene copolymers, fully hydrogenated styrenic polymers, polycarbonate PDMS copolymers, and polyolefins such as polyethylene, polypropylene, polymethyl pentene, polypropylene copolymers and cyclic olefin copolymers, and the like.

In some embodiments, the culture vessels (and/or units/compartments therein) contain a gas permeable, liquid impermeable film to allow transfer of gasses between a cell culture chamber and the exterior of the cell culture assembly. Such culture vessels can include spacers or spacer layers positioned adjacent the film, exterior to the chamber, to allow air flow between stacked units. One commercially available example of a cell culture apparatus containing such stacked gas permeable culture units is Corning's HYPERFLASK cell culture apparatus. Such cell culture units may be manufactured in any suitable manner, such as, for example, U.S. Pat. App. Ser. No. 61/130,421, entitled Assembly of Cell Culture Vessels, filed on May 30, 2008, which application is hereby incorporated herein by reference in its entirety to the extent that is does not conflict with the present disclosure. Examples of suitable gas permeable polymeric materials useful for forming a film include polystyrene, polyethylene, polycarbonate, polyolefin, ethylene vinyl acetate, polypropylene, polymethylpentene, polysulfone, polytetrafluoroethylene (PTFE) or compatible fluoropolymer, a silicone rubber or copolymer, poly(styrene-butadiene-styrene) or combinations of these materials. As manufacturing and compatibility for the growth of cells permits, various polymeric materials may be utilized. Preferably the film is of a thickness that allows for efficient transfer of gas across the film. For example, a polystyrene film may be of a thickness of about 0.003 inches (about 75 micrometers) in thickness, though various thicknesses are also permissive of cell growth. As such, the membrane may be of any thickness, preferably between about 25 and 250 micrometers, or between approximately 25 and 125 micrometers. The membrane allows for the free exchange of gases between the chamber of the assembly and the external environment and may take any size or shape. In some embodiments, the gas permeable film eliminates the need for an oxygenator, because oxygen can be transferred through the gas-permeable substrate from the surrounding environment. Preferably, the membrane is durable for manufacture, handling, and manipulation of the apparatus.

Embodiments of this disclosure include a unique housing or cabinet for containing one or more cell culture vessels with an interior cavity of the cabinet to create an optimized production system that has a compact footprint and minimizes manual handling and manipulation of the vessels. The cabinet is sized to house multiple cell culture vessels at once. In some embodiments, the multiple cell culture can be pre-configured or combined into a single unit, and the cabinet is sized to house one or more of these combined unit. In some embodiments, the cabinet is portable. For example, the cabinet can be equipped with wheels for easy handling and positioning. By being portable, the cabinet can be placed in a convenient location with a laboratory or production facility, and multiple cabinets can be positioned on compact arrays when it is convenient to do so (e.g., when manual interaction by the user is not needed).

To minimize manual handling of the cell culture vessels (or the combined units), the cabinet is designed to perform manipulations (e.g., reorientations or tilting operations) of the cell culture vessels that are needed during the culturing process. These manipulations can be semi- or fully-automated, or may involve manual actuation by the user. For example, the cabinet may include mechanical or electro-mechanical means (described herein) for tilting or reorienting the cell culture vessels while housed within the interior cavity. In some embodiments, the cabinet includes a lever that can be manually actuated by a user to tilt the entire cabinet, or at least a portion of the cabinet housing the cell culture vessels. This tilting operation can be performed during, for example, filling and emptying to facilitate those operations as needed for the multi-layered cell culture vessels described herein.

To distribute material such as, for example, cell culture media, buffers, proteolytic enzymes, etc. in the cell culture vessels, the cell culture vessel may be configured to tilt or be reoriented during operation. Orienting the vessel at one or more different positions can facilitate the culturing process of anchorage dependent, or adherent, cells within the vessel. According to various embodiments, the vessel may be equipped with a mechanism for reorienting or tilting the vessel while housed in the cabinet described herein. In some embodiments, the cabinet is configured to reorient or tilt the vessel. For example, the cabinet may be configured with a mechanical lifting means such as, e.g., an extending piston, a robotic arm, a lever, a tether, or a tilting shelf within the interior cavity of the cabinet. In some embodiments, a significant portion, majority, or entirety of the cabinet itself reorients or tilts, and thus reorients or tilts the one or more vessels stored within the cabinet. That is, according to various embodiments, the tilting of the vessel may cause the vessel's orientation to change with respect to the cabinet, or may maintain an relative orientation of the vessel to the cabinet in the case where the cabinet itself is being reoriented.

The possible orientations of the vessel may include one or more filling positions, one or more emptying positions, one or more culturing positions, etc. The one or more filling positions may be defined as positions operable to fill (e.g., effectively fill) the cell culture vessels, and likewise, the one or more emptying positions may be defined as positions operable to empty (e.g., effectively empty) the cell culture vessels. Further, one or more filling positions may exist since there may be different optimal filling positions for each stage of a filling cycle. For example, the cell culture vessels may be tilted at one or more particular, or selected, angles during the early stage of a filling cycle, and then tilted at one or more particular, or selected, angles during a later stage of the filling cycle different than those in the early stage to effectively fill the cell culture vessels. Still further one or more emptying positions may also exist since there may be different optimal emptying positions for each stage of an emptying cycle. For example, the cell culture vessels may be tilted at one or more particular, or selected, angles during the early part of an emptying cycle, and then tilted at one or more particular, or selected, angles during the latter part of the emptying cycle different than those in the early stage to effectively empty the cell culture vessels. The one or more culturing positions may generally include positions in which the cell culture, or growth, surfaces are parallel to a ground surface (e.g., to facilitate effective cell growth). Further, although a few different positions are described herein, fill positions, emptying positions, and incubation positions/conditions may be specific to the particular cell culture vessel used, and as such, the systems described herein may operate differently to accommodate the particular cell culture vessels being used. In other words, the fill positions, emptying positions, and incubation positions/conditions described herein are not the only positions that the systems described herein are capable of, and further, the systems described herein may be configured to accommodate the positions used for any particular cell culture vessel.

In some embodiments, the cell culture vessel and/or the cabinet is configured to move the cell culture vessels about a first axis and a second axis, each of the first axis and the second axis being perpendicular to one another and parallel to the ground surface (upon which cabinet is located). In embodiments, the cabinet may be configured to move the cell culture vessels vertically along a vertical axis to, for example, assist the loading and unloading of the cell culture vessels into or onto other various apparatus of the system for use thereof. In some embodiments, only a simple tilting of the cell culture vessel about a single axis is used.

The cell culture vessel may include at least one port, which can be fluidly connected to a fluid source that may be fed into a cell culture space of the vessel via gravity feed or a pumping apparatus. The cell culture vessel may further include a manifold fluidly coupling each cell culture module or unit of the cell culture vessel to at least one port such that materials can be pumped into and out of the cell culture vessel using the at least one port.

The pumping apparatus may be fluidly coupled to each of the cell culture vessels. In at least one embodiment, the pumping apparatus may include at least one pump for each of the cell culture vessels to, e.g., maintain a closed system, prevent cross contamination when using one pump for multiple cell culture vessels, etc. In other words, the pumping apparatus may include a plurality of pumps. Further, the pumping apparatus may include a plurality of valves which may be used to selectively connect, or fluidly couple, one or more reservoirs to the pumping apparatus such that materials located within the reservoirs may be pumped into the cell culture vessels and/or materials located within the cell culture vessels may be pumped into the reservoirs. Each reservoir may be defined as a fluid tight container, or vessel, configured to hold material. As used herein, “material,” e.g., that is pumped into and out of the cell culture vessel, may be defined as any flowable material (e.g., liquid) that may be used in cell culture processing. For example, material may include cell culture medium (e.g., containing cells to be cultured), spent medium, proteolytic enzymes, quench solutions, chelating solutions, buffers, transfection agents, etc.

The pumping apparatus and reservoirs may be coupled to the manipulation apparatus and/or any other portion of the cell culture system such that the pumping apparatus and the reservoirs may be integral, or self-contained, within the cell culture system.

A cell culture system described herein may further include a temperature control system. In embodiments, the temperature control system includes an incubation apparatus. The incubation apparatus may be generally described as any apparatus capable of incubating the cell culture vessels to facilitate incubation of the cells within the cell culture vessels. For example, the incubation apparatus may apply heat to the cell culture vessels in the range of 30 degrees Celsius to about 40 degrees Celsius. In at least one embodiment, the incubation apparatus may completely surround the cabinet. In at least another embodiment, the incubation apparatus may be apart from the cabinet such that the cabinet may be positioned or moved, with the cell culture vessels inside, into the incubation apparatus for incubation and/or out of the incubation apparatus after incubation. In some embodiments, the incubation apparatus is incorporated into the cabinet such that the cabinet controls the temperature within the interior cavity via the integrated temperature control or incubation apparatus.

The thermally-controlled environment can comprise an ambient temperature (i.e., the temperature of the environment surrounding the system), or controlled operating of, for example, from about 15 to about 50° C., 15 to about 45° C., 27 to about 45° C., 30 to about 40° C., and 35 to about 38° C., including intermediate values and ranges.

In embodiments where the cell culture system includes an incubator in which the cabinet and cell culture vessels are placed, the incubator can include one or more ports to enable the passage of tubing and/or wires (or other signal carriers for power sources and/or sensors). Accordingly, culturing operations of the cell culture vessels (e.g., filling and emptying) can be performed while the cabinet remains in the incubator. In some embodiments, one or more ports can allow for passage of connectors from the vessel sensors to the detectors located outside of the incubator, so that the detectors do not need to be made to withstand incubation temperatures and/or humidity.

As discussed above, embodiments of this disclosure include cell culture vessels having a gas-permeable film that provides a cell growth surface. In general, passive gas exchange may establish the appropriate dissolved gas concentrations in the cell growth medium to meet the metabolic needs of cells in culture. Cell culture mediums may rely on a carbonate/bicarbonate buffer system to interact with dissolved carbon dioxide to regulate the pH of the cell culture medium. This approach suffices for vessels that are placed in an incubator capable of controlling the carbon dioxide gas environment. However, for vessels placed in thermally controlled environments, such as incubators not having a controlled gas environment, laboratories, factories, and like accommodations, a method to successfully manage the pH is to alter the growth medium composition so that it is not regulated by carbon dioxide. Many cell culturists are reluctant to alter the growth media or buffer composition, so there can be a bias against using a gas permeable film vessel in a thermally controlled environment.

Although gas permeable film vessels (e.g., HYPERStack®, HYPERFlask®) are meant to provide users with a simple, passive gas diffusion system to supply cells with the oxygen required for metabolism, they do not function as well in an environment that does not contain 5% carbon dioxide gas if the cells are growing in medium with a carbonate/bicarbonate-based buffer system. Thermally controlled environments, such as warm rooms are large rooms maintained at the appropriate incubation temperature of 370° C., but they lack the humidification and gas control of typical incubators. Thermally-controlled environments are typically used for larger vessels such as the gas permeable HYPERStack-36 and 120 layered vessels, or the gas impermeable CellSTACK-10 and 40 layered vessels. Traditional gas impermeable stacked cell culture vessels such as the CellSTACK-40, or Cell Factory-40, have a headspace that permits the addition of carbon dioxide gas during or prior to incubation, so that they may still use a medium with a carbonate/bicarbonate-based buffer system in a thermally controlled room environment after gassing.

Since the prior art gas permeable film vessels do not have internal “headspace” like a traditional vessel, a 5% carbon dioxide environment can be provided to all the gas permeable films in the vessel by enclosing the entire body of the gas permeable film vessel in a gas impermeable enclosure. This enclosure can be constructed from any suitable material as long as it supports gas impermeability. The enclosure can be, for example, flexible, such as a poly-bag, or inflexible, such as a rigid-sided enclosure. The gas-impermeable enclosure can be, for example, a flexible sheet, a semi-rigid sheet having sealable ends, a rigid sheet having sealable ends, and like structures, or a combination thereof.

Those of ordinary skill in the art will appreciate the types of materials that are suitable for forming a gas-impermeable enclosure. For example, for a flexible bag-like enclosure, traditional materials such as polyethylene terephthalate can be relatively thin (e.g., 4 mil) but gas impermeable. However, polypropylene materials may need to have a greater thickness (e.g., 8 mil) to reduce the polypropylene's gas permeability. A laminated material can be used to offer certain properties, such as, for example, puncture resistance, strength at the heat seal, and excellent gas impermeability. While the flexible bag-like enclosure need not be optically clear, it may be a desirable feature for operators or users.

According to some embodiments, the gas-impermeable enclosure is sized to contain at least one cell culture vessel that has a gas permeable film, as described herein. A gas inlet port can be provided that passes through the gas-impermeable enclosure to provide gaseous communication between an external gas source and the cell culture vessel within the enclosure. In some embodiments, the system can also be provided with an exhaust port or pathway between the cell culture vessel and an external exhaust destination. The gas-impermeable enclosure can have a port for at least one sensor situated within or on the gas-impermeable enclosure to monitor the operation of the enclosed cell culture vessel. The ports can be, for example, air-tight sealable around a penetrating conduit, that is, the conduit can be, for example, a tube for carrying gas, a cable carrying an optic element, a wire carrying a signal, and like functional structures. Entry ports can be secured in the walls of the gas impermeable enclosure bag to permit connection of tubing for active gas exchange in- and out-of the enclosure, and for example optional connections for fiber optic cable positions to various optical sensors.

Apertures or openings can also be present in the bag enclosure to permit the vessel ports for air and liquid-handling to protrude (e.g., a manifold connection). The apertures can be, for example, elasticized to create an air-tight seal around the ports, or they may be sealed by the tubing or O-rings that can be added to the ports after the bag enclosure is positioned over the vessel. The enclosure bag can be slid over the vessel and the enclosure bag can be cinched at, for example, the base using, for example, a drawstring, cable tie, or like other fastening means or method to secure the bag enclosure around the vessel. Alternatively, the bag enclosure can have, for example, a zipper, a hook-and-loop fabric (e.g., Velcro®) or magnetic closure to permit wrapping and securing the bag enclosure around the exterior of the vessel. Less flexible gas-impermeable enclosures are also possible and may be constructed, for example, from preformed parts (e.g., molded, thermoformed, extruded stock, etc.) that are appropriately sized to enclose the enclosed vessel, and with which assembled parts can be secured. For example, a tube structure comprising an air-tight body, base plate, removable lid or cap, and the aforementioned communication and connection ports and apertures. The preformed enclosure assembly or the lid, or cap of the enclosure can contain the abovementioned gas and sensing ports and connections called for by the abovementioned flexible enclosure.

In some embodiments, the system is provided with a controller, and at least one of the source of gas, the exhaust gas pathway, and the at least one sensor are in communication with the controller to control the gaseous properties (e.g., gas composition and concentrations) within the gas-impermeable enclosure. The controller can be disposed external to the gas-impermeable enclosure and/or external to a cabinet enclosing the cell culture vessel, or may be integrated into one of more of the gas-impermeable enclosure or cabinet.

The source of gas can be, for example, at least one of: carbon dioxide (CO₂), carbon dioxide balance air, oxygen (O₂), water vapor (humidity), and like substances, or a combination thereof. The at least one sensor can be, for example, at least one sensor for: carbon dioxide, oxygen, pH, humidity, or a combination thereof. The relative percentages of carbon dioxide, oxygen, humidity, or a combination thereof, within the gas impermeable enclosure can be, for example, from about 1 to 35% carbon dioxide, from about 1 to 50% oxygen, and from about 1 to 95% humidity. The pre-determined ranges of values can include, for example, one or more of carbon dioxide from about 1 to about 10%; oxygen from about 1 to about 30%; relative humidity from about 10 to about 95%; and acidity (pH) from about 4 to about 9.

Embodiments of this disclosure include methods of using a cell culture system described herein, including those with a gas-impermeable enclosure. The method can include monitoring, for example, the concentration or activity of at least one of a gas, a gas mixture, humidity, acidity (pH), or a combination thereof delivered to the gas permeable film vessel having a cell culture contained in the cell culture system, and adjusting the at least one of the gas, gas mixture, humidity, acidity (pH), or a combination thereof. If the monitoring indicates an excursion from a pre-determined range of values, the controller adjusts at least one of the gas, the gas mixture, the humidity, the acidity (pH), or a combination thereof, to return the system to the pre-determined range of values.

According to embodiments of the present disclosure, the spatial and handling requirements of high-yield cell culture vessels are decreased by providing high-yield culturing systems in compact footprints with handling that can be automated or semi-automated. The systems and methods of this disclosure can include the cell culture apparatus, enclosures (e.g., cabinets and/or gas-impermeable barriers or enclosures), sensors, fluid sources, connections, and pathways (e.g., tubing, fittings, manifolds, gas and media sources, exhaust outlets, etc.), thermal or temperature control systems, pumping systems, and control systems.

The cell culture apparatus includes at least one cell culture vessel, cabinet apparatus, pumping apparatus, monitoring apparatus, and control apparatus. The at least one cell culture vessel is configured to culture cells using a plurality of parallel cell culture surfaces and the at least one cell culture vessel includes at least one port configured to allow material to flow into and out of the at least one cell culture vessel. The cabinet is configured to hold the at least one cell culture vessel within an interior cavity. The cabinet is further configured to rotate the at least one cell culture vessel. The type and degree of rotation can vary depending on the design of the cell culture vessel, and the stage of culturing during which the vessel is being rotated. In some embodiment, the rotation is a simple rotation about a single axis, but in other embodiments, it can be a complex rotation about a first rotation axis and about a second rotation axis (e.g., where the first rotation axis is perpendicular to the second rotation axis, and where each of the first rotation axis and the second rotation axis are parallel a ground surface). The pumping apparatus is fluidly coupled to the at least one port of the at least one cell culture vessel and is configured to pump material into and out of the at least one cell culture vessel through the at least one port. The monitoring apparatus is configured to monitor one or more parameters of the at least one cell culture vessel, the cabinet, and the pumping apparatus. The control apparatus is operably coupled to the cabinet, the pumping apparatus, and the monitoring apparatus, and is configured to coordinate movement of the at least one cell culture vessel using the rotation operation of the cabinet with the pumping of material into and out of the at least one culture vessel using the pumping apparatus.

In various embodiments, the control apparatus is further configured to monitor, using the monitoring apparatus, one or more parameters of the at least one cell culture vessel, the cabinet, and the pumping apparatus, and adjust one or more parameters of the at least one cell culture vessel, the cabinet, and the pumping apparatus based on the monitored one or more parameters.

A cell culture system described herein may include monitoring apparatus. Generally, monitoring apparatus may be configured to monitor any one or more parameters associated with the cell culture system. For example, the monitoring apparatus may be configured to monitor one or more of the cell culture vessels, the cabinet, the pumping apparatus, the reservoirs, the cell release apparatus, the incubation apparatus, etc. Further, the monitoring apparatus may include position sensors, temperature sensors, pressure sensors, light sensors, fill position sensors, oxygen sensors, carbon dioxide sensors, pH sensors, gas concentration sensors, fluorescent-imaging based sensors, optical sensors, glucose sensors, lactate sensors, ammonium sensors, load cells (e.g., for weighing cell culture vessels), electrical impedance sensors, ultrasonic impedance sensors, vision systems, and/or any other sensor that may be used in the cell culture system. The monitoring apparatus may be used by the control apparatus of the cell culture system to monitor the cell culture system to provide feedback for adjusting one or more parameters with respect the cell culture system. The cell culture vessels and modules of this disclosure can incorporate sensors for detecting cell confluence and monitoring metabolites. In some embodiments, Raman probes can be used, and sensors for the Raman probes can be positioned on an exterior of the cabinet.

The control apparatus of the cell culture system may include one or more computing devices capable of processing data. The control apparatus may include, e.g., microprocessors, programmable logic arrays, data storage (e.g., volatile or non-volatile memory and/or storage elements), input devices, output devices, etc. The control apparatus may be programmed to implement the methods or portions of the methods described herein and may be operably coupled to each element of the cell culture system to, e.g., monitor or adjust one or more parameters with respect to each element of the cell culture system. For example, the control apparatus may be operably coupled to the cell culture vessels, the manipulation apparatus, the pumping apparatus, the reservoirs, the cell release apparatus, the incubation apparatus, or the monitoring apparatus.

As described herein, “operably coupled” may be defined as connected (e.g., wired or wirelessly) such that information (e.g., image data, commands, etc.) may be transmitted between each object.

In embodiments, position sensors of the monitoring apparatus may be configured to monitor the position of the cell culture vessels and/or the position of the cabinet or support surfaces within the cabinet, such as, e.g., the rotation of the cell culture vessels about a first axis parallel a ground surface, the rotation of the cell culture vessels about a second axis parallel to ground surface, the distance of the cell culture vessels above the ground surface, etc. Such position data may be used, e.g., by the control apparatus, to confirm movements made to the cell culture vessels during culturing. In at least another embodiment, temperature sensors of the monitoring apparatus may be configured to monitor the temperature inside or outside of the cell culture vessels and/or the cabinet, and/or the temperature inside the incubator apparatus. Such temperature data may be used for monitoring purposes and/or adjusting of the incubator apparatus.

In embodiments, pressure sensors of monitoring apparatus may be configured to measure the pressure within each cell culture vessel or module, each reservoir, and/or the incubation apparatus. In at least one embodiment, fill level, or position, sensors of the monitoring apparatus may be configured to monitor the amount of material (e.g., the fill level) within the cell culture vessels or the reservoirs. Such fill level data may be used to determine if the cell culture vessels are full. In at least one embodiment, oxygen sensors of the monitoring apparatus may be configured to monitor the oxygen concentration within the cell culture vessels, the gas-impermeable enclosure, the cabinet, the reservoirs, or the incubation apparatus, and carbon dioxide sensors of the monitoring apparatus may be configured to monitor the carbon dioxide concentration within the cell culture vessels, the gas-impermeable enclosure, the cabinet, the reservoirs, or the incubation apparatus. In embodiments, the control apparatus may be configured to modify the rate at which material is pumped into and out of each culture vessel using the pumping apparatus based on the one or more monitored parameters of the culture vessels.

In embodiments, the optical sensors of the monitor apparatus may be configured to image the material within the cell culture vessels (e.g., image the cell culture medium, etc.) and the control apparatus may be configured to provide the images to a user. Further, the user may be remote from the system, e.g., such that the user can view images of the cell culture without being located local, or near, the system. In other words, the cell culturing system can provide remote visualization of the cell culture (e.g., which may provide rapid assessment of cell confluence). Further, after the cells are released from the cell culture surfaces, the cells could be also checked using such remote visualization. In effect, optical sensors of the monitoring apparatus may provide a remote microscope to view the cell cultures.

The cell culture systems described herein may provide a semi- or fully-automated solution to accomplish one or more processes used to seed, grow and harvest adherent cells from stacked cell culture vessels. Further, the systems described herein may bring together multiple discrete components and integrate\them with one central computer-controlled machine that may use one or more sensing devices to provide feedback to the cell culturist or user. Further, one or more cell culture systems described herein may include a Human Machine Interface (HMI) that allows users to enter numeric process variables that are specific to their cell culture needs, full computer or Programmable Logic Control (PLC) to control one or more cell culturing parameters such as, e.g., the fill rate, fill pressure and fill volume of each cell culture vessel, semi-of fully-automated positioning of vessels coordinated with pump speed adjustment during filling/emptying, equilibration, and cell removal phases, manually controlled or semi- or fully-automated valves to control the flow of media into and out of the vessel, proper positioning of vent filters to avoid wetting, automated pressure testing to ensure vessel integrity, integrated safety features, and process monitoring of time, temperature, pH, gas concentrations and metabolites.

Embodiments of the cell culture systems described herein may include monitoring apparatus such as, e.g., one or more sensors, that may be configured to detect the flow rates, fill volumes, temperatures, pressures, etc. with respect to the cell culture vessels. In at least one embodiment, the cell culture systems may be configured to apply and monitor pressure in a cell culture vessel to ensure vessel integrity. Further, in at least one embodiment, the cell culture systems may incorporate, or include, control of temperature and gas concentrations within and/or around the cell culture vessels.

Still further, in various embodiments, the cell culture systems described herein may include one or more human machine interfaces that may be configured to permit a human operator to monitor and adjust automated processes as well as monitor cell culture conditions such as pH, gas concentrations, metabolites, temperature, etc. Further, such human machine interfaces may be located remotely, e.g., such that the physical presence of a human operator local to the system may not be required.

A cell culture system may further include cell release apparatus. Generally, cell release apparatus may be operable to release cells adhered, attached, or anchored to the cell culture, or growth, surfaces of the cell culture vessels, e.g., after the cells have been cultured. In at least one embodiment, the cell release apparatus may include shaking apparatus configured to shake the cell culture vessels at a frequency greater than or equal to about 0.1 kHz, about 0.5 kHz, about 1 kHz, etc. and/or less than or equal to about 5 kHz, about 10 kHz, about 15 kHz, about 20 kHz, etc. to release at least a portion of a plurality of cells adhered to the cell culture surfaces of cell culture vessels. In at least one embodiment, the cell release apparatus may include shaking apparatus configured to shake the cell culture vessels at an amplitude of about 12 millimeters (mm) to about 26 mm. Further, the shaking path can be oriented at a wide range of angles relative to the cell culture surfaces of the cell culture vessels. For example, the shaking apparatus may be configured to move the cell culture vessels 12 in a circular path, vertically, parallel to the cell culture surfaces, and in linear reciprocation. The shaking apparatus may be as described in U.S. Prov. Pat. App. Ser. No. 61/527,164 entitled “METHODS OF RELEASING CELLS ADHERED TO A CELL CULTURE SURFACE” and filed Aug. 25, 2011, which is incorporated herein by reference in its entirety to the extent it does not conflict with the disclosure presented herein.

The shaking apparatus may be integral or separate from the cabinet. For example, the shaking apparatus may be coupled to the cabinet and configured to shake at least a portion of the cabinet such that the cell culture vessels held by the cabinet may shake. In some embodiments, the shaking apparatus can be contained within the cabinet and configured to shake the cell culture vessels within the cabinet, without shaking the entire cabinet. For example, support surfaces or shelves can be provided in the interior cavity of the cabinet, and the shaking apparatus can include those shelves or an apparatus provided on a shelf. Further, for example, the shaking apparatus may be located apart, away, or separately from the cabinet. In this example, the cabinet or the shaking apparatus may move with respect to the other to locate the shaking apparatus and cell culture vessels in contact with one another such that the shaking apparatus can shake the cell culture vessels to release at least a portion of a plurality of cells adhered to the cell culture surfaces of the cell culture vessels. In embodiments, the shaking apparatus may be configured to be in contact with, or in close proximity to, at least a portion of the cell culture vessels and slide across, or relative to, the cell culture vessel so as to deliver shaking energy to portions of the cell culture vessel as the transducer slides relative to the vessel.

In embodiments, the shaking apparatus may include a platform upon which the cell culture vessels may be placed within the cabinet. After the cell culture vessels have been placed on the platform, the platform may shake thereby shaking the cell culture vessels to release at least a portion of a plurality of cells adhered to the cell culture surfaces of the cell culture vessels.

In embodiments, cell release apparatus may include ultrasonic transducer apparatus configured to provide ultrasonic energy to the cell culture vessels at a frequency greater than or equal to about 1 kHz, about 10 kHz, about 15 kHz, etc. and less than or equal to about 20 kHz, about 30 kHz, about 40 kHz, etc. Further, the ultrasonic transducer apparatus may be configured to provide ultrasonic energy to the cell culture vessels for about 5 seconds to about 30 seconds for each cell culture vessel one or more times. For example, the ultrasonic transducer apparatus may be as described in U.S. Pat. App. Pub. No. 2009/0298153 entitled “METHOD FOR ULTRASONIC CELL REMOVAL,” published on Dec. 3, 2009, and filed on May 19, 2009, which is also incorporated herein by reference in its entirety to the extent that it does not conflict with the disclosure presented herein. Further, ultrasonic transducer apparatus may be configured to be movable with respect to the cell culture vessels and/or the cabinet so as to be able to deliver ultrasonic energy to at least one of the one or more chambers, units, modules, or compartments of the cell culture vessels. For example, the ultrasonic transducer apparatus may be configured to be in contact with, or in close proximity to, at least a portion of the cell culture vessels and slide across, or relative to, the cell culture vessel so as to deliver ultrasonic energy to portions of the cell culture vessel as the transducer slides relative to the vessel. Further, for example, the ultrasonic transducer apparatus may be configured to be directional such that it may direct, or sweep, the ultrasonic energy across the cell culture vessel, using, e.g., a horn.

Embodiments of this disclosure include methods of using cell culture systems described herein. The method may include manipulating one or more cell culture vessels and transferring material into or out of the one or more cell culture vessels either during or after the manipulation. For example, the cell culture vessels may be manipulated into a filling position, and after being manipulated to the filling position, the method may initiate the transfer of materials such as, e.g., cell culture medium, etc., from one or more reservoirs to the cell culture vessels. Further, for example, the cell culture vessels may be manipulated into an emptying position, and after being manipulated into the emptying position, the method may initiate the transfer of materials such as, e.g., spend medium, harvested cells, etc., from the cell culture vessels to one or more reservoirs.

Filling and emptying methods for use with cell culture systems described herein may include multiple different positions to facilitate the filling and emptying methods. As such, the method to continually, periodically, or on an as-needed basis, manipulate the cell culture vessels (e.g., into one or more positions) while transferring materials to/from the cell culture vessels (as shown by the arrow looping back from process to process). In at least one embodiment, the cell culture vessels may be manipulated and materials may be transferred at the same time. In embodiments, one or more sensor may detect fill level of the culture vessel to provide feedback to a control unit for purposes of manipulating the vessel to the appropriate position during the filling or emptying process. Any suitable sensor may be used to detect fill level of the vessel during the filling or emptying process. In embodiments, a load sensor or other mass sensor may be used to measure the mass of each of the at least one cell culture vessel, e.g., for purposes of detecting fill level (e.g., the data from the load sensor or other mass sensor may be used in coordinating the movement of the at least one cell culture vessel and/or the cabinet with the pumping of material into and out of the at least one culture vessel using the pumping apparatus). In embodiments, one or more optical sensor, infrared sensor, or the like may be suitably positioned along the culture vessel to detect fill level.

The present disclosure describes cell culture systems that enable semi- or fully-automated filling and/or emptying of cell culture vessels with liquid media. It is contemplated that embodiments of the system can include different combinations of various components discussed herein, including one or more of a cell culture vessel; a storage cabinet; a fill sensor for detecting a fill level of the cell culture vessel; an actuator for changing an orientation of the cell culture vessel and/or the cabinet during filling; a controller; a pressure sensor; and various connections, fittings, tubing, and manifolds. Embodiments described herein use fill sensors for monitoring the level of liquid media during filling or emptying of the vessel and reorient the cell culture vessel or adjust the filling rate depending on the fill level. The systems and methods disclosed herein can enable semi-automated or fully-automated filling and/or emptying of a cell culture vessel. As a result, cell culture systems and methods are provided that lower the risk of leaks, contamination, and other stresses on the cell culture system, and the degree of user monitoring and attention needed during the filling or emptying procedure is decreased.

In embodiments of this disclosure, one or more sensors can be used to measure a fill level in the cell culture vessel or the manifold. The filling rate across cell culture devices can vary somewhat, so if a user is attempting to fill multiple vessels at once, the sensor on each cell culture apparatus can determine the appropriate time for each specific vessel to be re-oriented or for fluid flow to be changed.

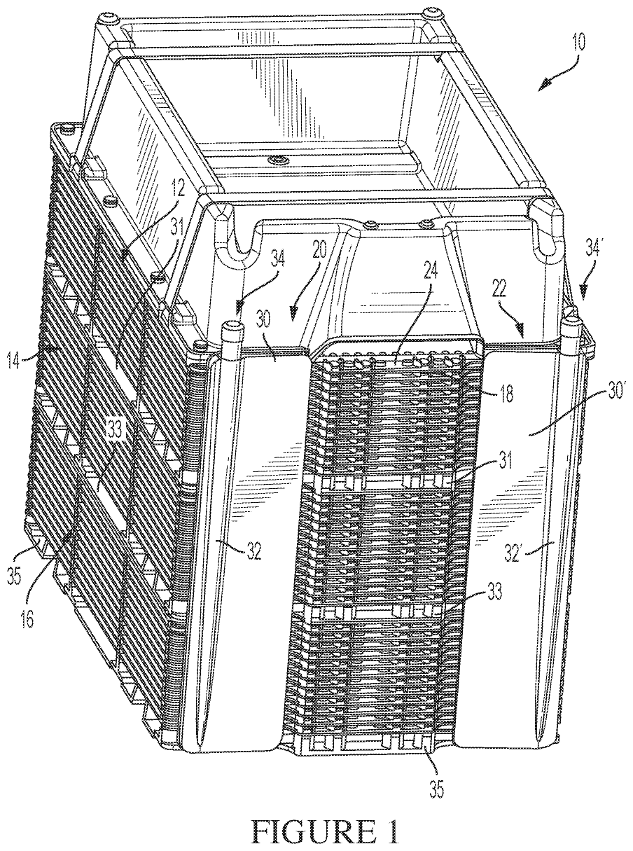

Referring to FIG. 1, a cell culture apparatus 10 includes three cell culture modules 12, 14, and 16, each containing multiple layers of cell culture chambers 18, and are stacked, one on top of the other, to form the multiple layer cell culture apparatus 10. Each cell culture module 12, 14, and 16 utilizes two manifolds 20 and 22. Liquid may enter and exit the cell culture modules 12, 14, and 16 through the first manifold 20. Thus, the first manifold 20 may be referred to as a fluid manifold. Air may enter and exit the cell culture modules 12, 14, and 16 through the second manifold 22. Thus, the second manifold 22 may be referred to as an air manifold.

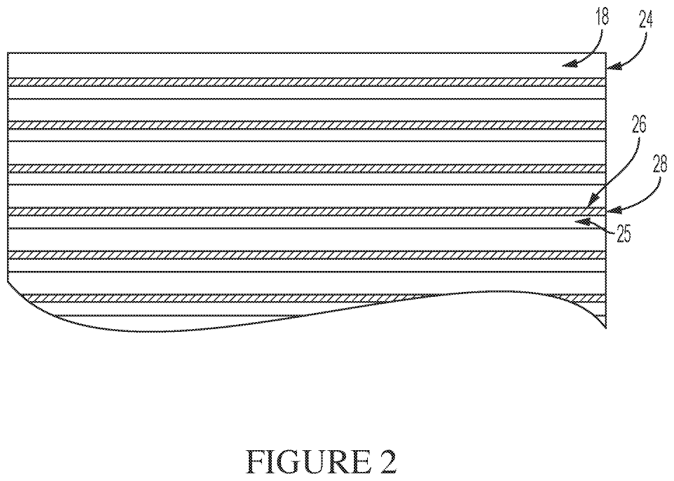

The cell culture modules 12, 14, and 16 may each include multiple stackette layers 24 that, when stacked together, form the multiple cell culture chambers 18 having tracheal spaces (air spaces) 25 there between, as shown in FIG. 2. FIG. 2 is a schematic representation of the multiple stackette layers 24 that are stacked together to form the layered cell culture chambers 18 and cell culture surfaces 26 that include a gas permeable, liquid impermeable film 28, for example, the stackette layers 24 include the tracheal spaces 25 to allow transfer of gasses between the cell culture chambers 18 and the exterior of the cell culture apparatus 10. Referring back to FIG. 1, the cell culture modules 12, 14, and 16 may be separated from one another by spacers 31, 33, and 35. The spacers 31, 33, and 35 can provide structural support for the individual cell culture modules 12, 14, and 16. In some embodiments, spacers 31 and/or 33 may be replaced by additional stackette layers 24 to provide a higher total number of cell culture chambers 18. Further, a riser volume may be provided above the cell culture module 12 to catch residual air, rather than air residing in the cell culture chambers 18.

In some embodiments, the culture modules contain the gas permeable, liquid impermeable film 28 to allow transfer of gasses between the cell culture chamber 18 and ultimately with the exterior of the cell culture vessel. Such culture modules can include spacers or spacer layers positioned adjacent the film, exterior to the chamber, to allow air flow between stacked units. One commercially available example of a cell culture apparatus containing such stacked gas permeable culture units is Corning's HYPERStack™ cell culture apparatus.

As mentioned above, the cell culture modules 12, 14, and 16 may be connected together using the manifolds 20 and 22. The manifold 20 includes a side wall base structure 30 and a column structure 32 that is formed as a monolithic part of the side wall base structure 30 providing a unitary manifold 20. The column structure 32 includes a barb structure 34 and provides at least part of a fluid flow pathway from the barb structure 34 that is in fluid communication with the individual cell culture chambers 18 within the cell culture modules 12, 14, and 16. The manifold 20 may be configured to allow filling and emptying of the cell culture chambers 18.

The manifold 22 also includes a side wall base structure 30′ and a column structure 32′ that is formed as a monolithic part of the side wall base structure 30′ providing a unitary manifold 22. The column structure 32′ includes a barb structure 34′ and provides at least part of a fluid flow pathway from the individual cell culture chambers 18 within the cell culture modules 12, 14, and 16 to the barb structure 34′. The manifold 22 may be configured to allow filling and emptying of the cell culture chambers 18 by allowing air to enter and exit the cell culture apparatus 10. In some embodiments, the column structure 32′ may be offset from the illustrated location in order to control media flow into the column structure 32′.

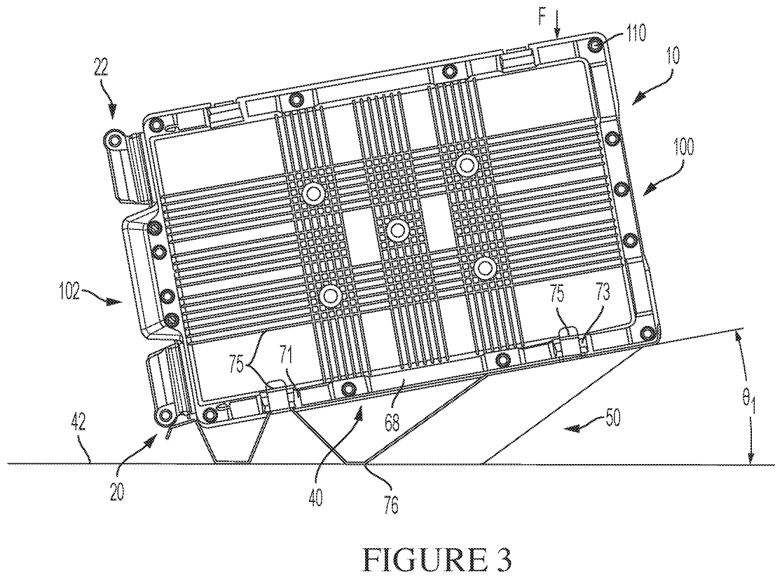

For a typical filling procedure, a cell culture apparatus 10 can be placed with its left side down, facing a support surface or tray. In this orientation, the front of the cell culture apparatus 10 with manifolds 20 and 22 is tilted downward for a first filling orientation at the start of filling (see the side view of FIG. 3). The flow of liquid media into the cell culture vessel is then initiated. For example, the media can be pumped into the lower column structure 32 through the barb structure 34 with the use of a peristatic pump, or the vessel can be filled with gravity induced flow. As the liquid media within the cell culture apparatus 10 and rises to a first fill level at a predetermined position, the cell culture apparatus 10 (and filling tray, if used) is re-oriented to a second filling position. In this second filling orientation, filling can continue until the liquid media reaches a final fill level in the cell culture apparatus 10. Upon reaching the final fill level, the flow of media is stopped and the inlet and outlet from the manifolds 20, 22 can be shut or clamped off to close the system. At this time, the cell culture apparatus 10 is ready for cell culture use.

Embodiments of this disclosure include a type of filling tray or multi-position support, as mentioned above. It is contemplated that this multi-position support can be integrated into the support surfaces within the interior cavity of the cabinet. For example, the multi-position support can be placed on the support surface, or the support surface can take the form of the multi-position support. Embodiments are not limited to using the multi-position support shown. However, the multi-position support is discussed below and shown in the accompanying figures to illustrate the tilting operation of cell culture vessels according to some embodiments. Further details of the multi-position support can be found in U.S. Provisional Patent Application No. 63/056,913 filed Jul. 27, 2020, the disclosure of which is incorporated herein by reference.

Referring to FIG. 3, using the multi-position support 50, the cell culture apparatus 10 may be filled and emptied with the cell culture apparatus lying on a side 40 and tilted, as illustrated by FIG. 3. The cell culture apparatus 10 may be reliably positioned on the side 40 at a predetermined tilt angle θ₁ (e.g., between about 10 degrees and about 12 degrees) relative to a support member 42 or horizontal using a multi-position support 50. The side 40 closest to the fluid manifold 20 is placed on the multi-position support 50 such that the fluid manifold 20 is lower than the air manifold 22. As will be described in greater detail below, the multi-position support 50 can be tilted between an upright configuration (as shown by FIG. 3) and a tilted configuration to position the cell culture apparatus 10 at different angles relative to horizontal.

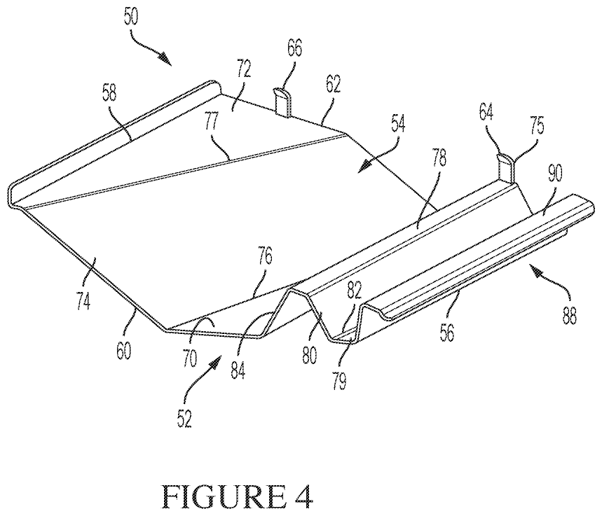

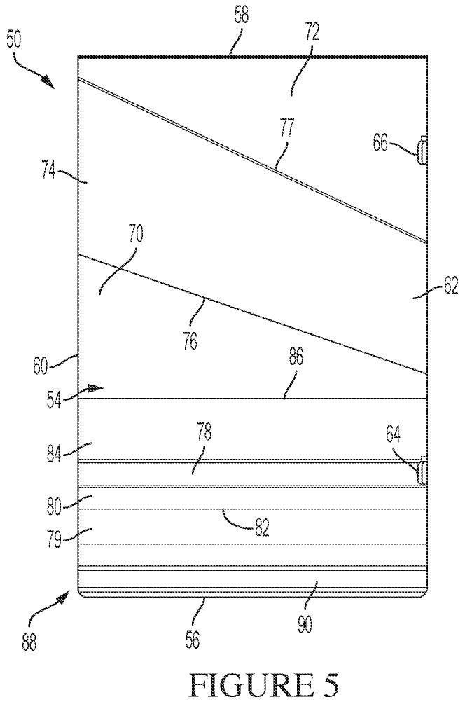

Referring to FIGS. 4 and 5, the multi-position support 50 is illustrated in isolation and is formed as a monolithic, bent plate that includes a bottom 52, a top 54, opposite ends 56 and 58 and opposite sides 60 and 62. At side 62, the multi-position support 50 includes location tabs 64 and 66 that engage a bottom edge 68 of the cell culture apparatus 10 (FIG. 3) with the multi-position support 50 in an upright, standing position and helps to hold the cell culture apparatus 10 in place on the multi-position support 50. In some embodiments, the bottom edge 68 of the cell culture apparatus 10 may be provided with recessed features 71 and 73 that are sized and located to receive the location tabs 64 and 66. The location tabs 64 and 66 may include a bend 75 that can be used to grip the bottom edge 68 and inhibit side-to-side movement of the cell culture apparatus 10 off of the multi-position support 50.

The multi-position support 50 includes a primary base 70 that rests against a support member (e.g., a table or lab bench) with the multi-position support 50 in an upright configuration as shown. A primary support surface 72 is provided that is offset vertically from the primary base 70 in the upright configuration and supports the cell culture apparatus 10 thereon. The multi-position support 50 further includes an intermediate surface 74 that extends between the primary base 70 and the primary support surface 72. The intermediate surface 74 meets the primary base 70 at an interface 76 formed as a bend that extends at an oblique angle to the sides 60 and 62 of the multi-position support 50. The intermediate surface 74 also meets the primary support surface 72 at an interface 77 formed as a bend that extends at an oblique angle to the sides 60 and 62. In some embodiments, the oblique angles of the interfaces 76 and 77 are about the same (e.g., within five degrees) relative to the sides 60 and 62 or they may be different.

The multi-position support 50 further includes a secondary base 79 that rests against the support member with the multi-position support 50 in the upright configuration. A secondary support surface 78 is provided that is offset vertically from the secondary base 79 in the upright configuration and supports the cell culture apparatus 10 thereon. The secondary support surface 78 and the primary support surface 72 lie in a same plane that is at an angle to horizontal and is also oblique to the primary base 70 and the secondary base 79. The multi-position support 50 further includes another intermediate surface 80 that extends between the secondary base 79 and the secondary support surface 78. The intermediate surface 80 meets the secondary base 79 at an interface 82 formed as a bend that extends perpendicular to the sides 60 and 62 of the multi-position support 50. Another intermediate surface 84 extends between the primary base 70 and the secondary support surface 78. The intermediate surface 84 meets the primary base 70 at an interface 86 formed as a bend that also extends perpendicular to the sides 60 and 62. A handle feature 88 is provided at the end 56. The handle feature 88 can also include a support flange 90 that is offset vertically from the secondary base 79 in the upright configuration and supports the cell culture apparatus 10 thereon. The end 58 is provided with a support flange 94 that extends vertically outward from the primary support surface 72 and is used to hold the cell culture device 10 on the primary support surface 72.

FIG. 3 illustrates the multi-position support 50 with the cell culture apparatus 10 supported thereon in the upright configuration. In the upright configuration, the cell culture apparatus 10 has a rear 100 that is more elevated than a front 102 at the angle θ₁ (between 10 degrees and 12 degrees) to horizontal. However, the top to bottom angle is parallel (zero degrees) to horizontal. This upright configuration may place the cell culture apparatus 10 in an initial fill position to begin filling the cell culture apparatus 10 where the front 102 is lower than the rear 100 thereby providing a gentler fill angle, which can reduce foaming in the fluid and promote air evacuation through the air manifold and filter connected thereto.

As the cell culture apparatus 10 is being filled with the multi-position support 50 in the upright configuration, the fluid level within the cell culture apparatus 10 rises toward the air manifold 22 and toward the filter that is connected to the air manifold. Wetting of the filter can reduce airflow rate out of the cell culture apparatus 10 thereby pressurizing the interior, which can lead to an undesirable environment within the cell culture apparatus 10. To reduce a likelihood that fluid reaches the filter, the multi-position support 50 is provided with a tilted configuration where the multi-position support 50 along with the cell culture apparatus 10 is rotated without lifting either the multi-position support 50 or the cell culture apparatus 10. The multi-position support 50 along with the cell culture apparatus 10 can simply be manually tilted by applying a force F to a rear corner 110 of the cell culture apparatus 10, which causes the multi-position support 50 and the cell culture apparatus 10 to rotate about the interface 76. Because the interface 76 extends at the oblique angle to the sides 60 and 62 of the multi-position support 50, the tilting changes both the front to rear angle and the top to bottom angle to increase the elevation of a top of the air manifold where the filter is connected. According to embodiments described below, this tilting operation can also be performed by an automated cell culture system, without the manual application of the force F. However, the same multi-position support 50 shown and described can be used for both the manual and automated tilting.

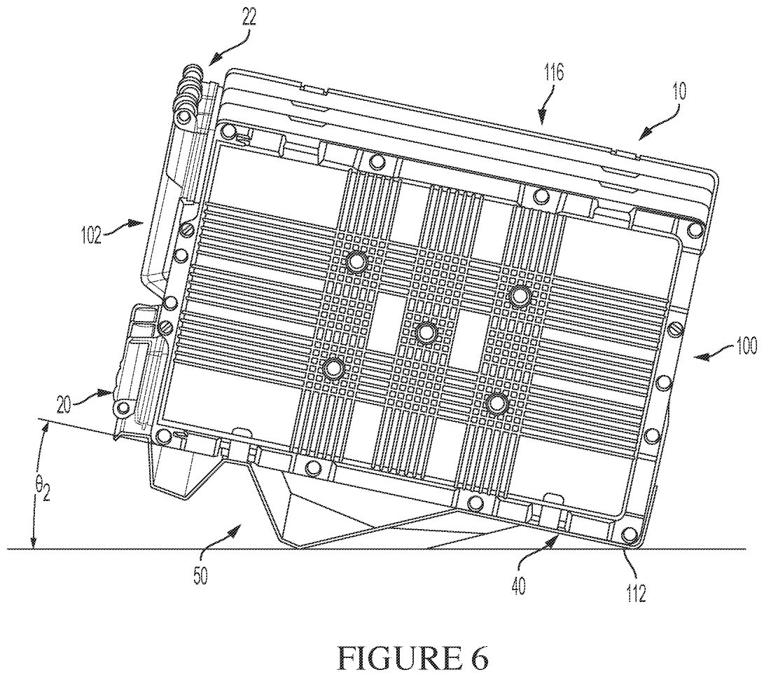

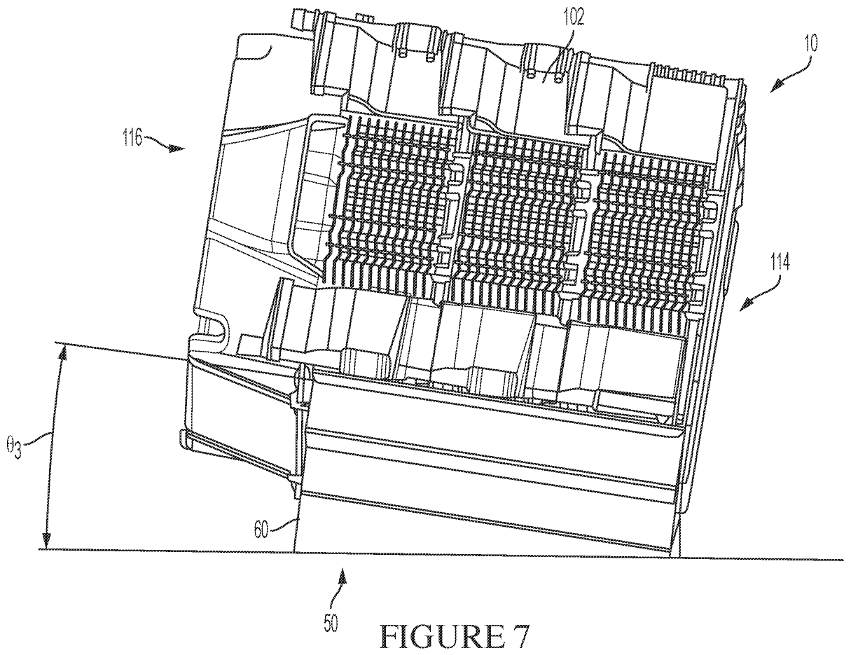

Referring to FIG. 6, the multi-position support 50 and the cell culture apparatus 10 are shown in the tilted configuration where the front 102 is now more elevated than the rear 100 providing an angle θ₂ (between 11 degrees and 13 degrees) to horizontal. As can be seen, a corner 112 between the side 40 and the rear 100 of the cell culture apparatus 10 rests against the support member in the tilted configuration. Referring to FIG. 7, a top 116 is more elevated than the bottom 114 at an angle θ₃ (between seven degrees and nine degrees) to horizontal. The tilted configuration thereby provides the multi-position support 50 and the cell culture apparatus 10 with the compound angle of both θ₂ (front to rear) and θ₃ (top to bottom), which may be referred to as an end fill position. Once the cell culture apparatus 10 is filled, the side 60 of the multi-position support 50 nearest the top 116 of the cell culture apparatus 10 may be rotated upward until the cell culture apparatus 10 is in an upright, standing position. Thus, the cell culture apparatus 10 can be manipulated throughout the fill process using only the multi-position support 50 without any need for lifting the cell culture apparatus 10 from the multi-position support 50. Emptying the cell culture apparatus 10 can be performed in a reverse order.

The above-described multi-position supports can be used to manipulate cell culture apparatuses without any need for handling the cell culture apparatuses separately from the multi-position supports during a fill or empty operation. The multi-position supports can thereby increase process efficiency and save users time due to higher fill and empty rates as well as from simple quick angle change procedures. The multi-position supports may further provide clear and concise control protocols which can reduce errors, reduce the possibility of product failure and/or damage, reduce angle variations due to method cradling using the multi-position supports and fixed tilt angles. Providing the multi-position supports with a compound tilt angle reduces the change of wetting out the filter attached to the air manifold. In some embodiments, the multi-position devices may be formed of stainless steel, which can provide increased durability and meet good manufacturing practices (GMP). The multi-position apparatuses may be formed from a sheet material on a metal brake to reduce manufacturing costs. Modifications can be made without incurring significant costs for re-tooling.

The reorientation of the multi-position support can be performed manually in some embodiments. In other embodiments, the tilting of the multi-position support is automated and controlled by the control system of the cell culture system described above.

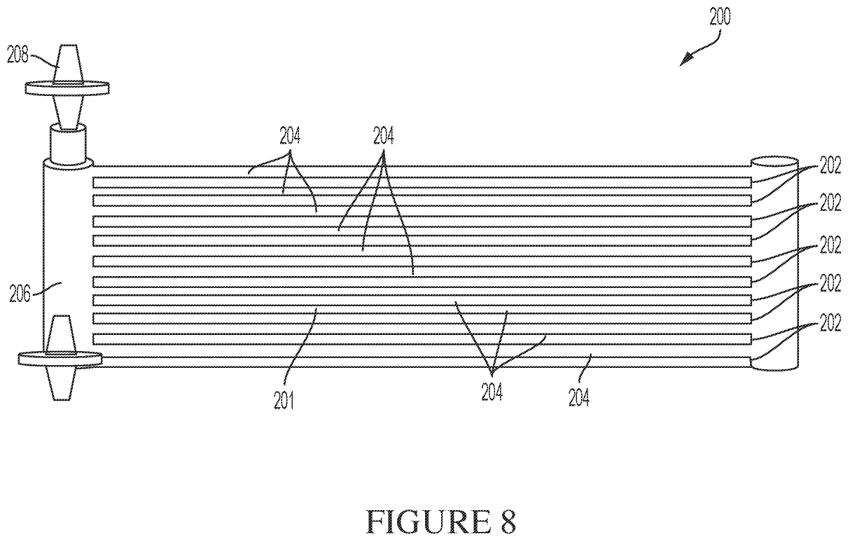

FIG. 8 is a cross-section view of a cell culture vessel 200 according to another embodiment. Similar to the embodiment shown in FIGS. 1 and 2, the vessel 200 is a multi-layered cell culture vessel. In FIG. 8, the vessel 200 contains a cell culture space 201 shown with 10 cell culture layers 202; however, it is understood that embodiments can include vessels with greater or fewer numbers of layers. Each layer includes a polymeric support surface (layer 202) for growing anchorage-dependent, or adherent, cells, and a gas permeable film 204. The vessel 200 is shown in a state of being filled with liquid media 206 used during the cell culturing process. The vessel 200 can be provided with a two-dimensional surface on the layers 202 for 2D cell culture. If used for static cell culture (as opposed to perfusion cell culture), the vessel 200 can be provided with a vent 208, which allows, for example, exhaust gas to escape the culture space within the vessel 200.

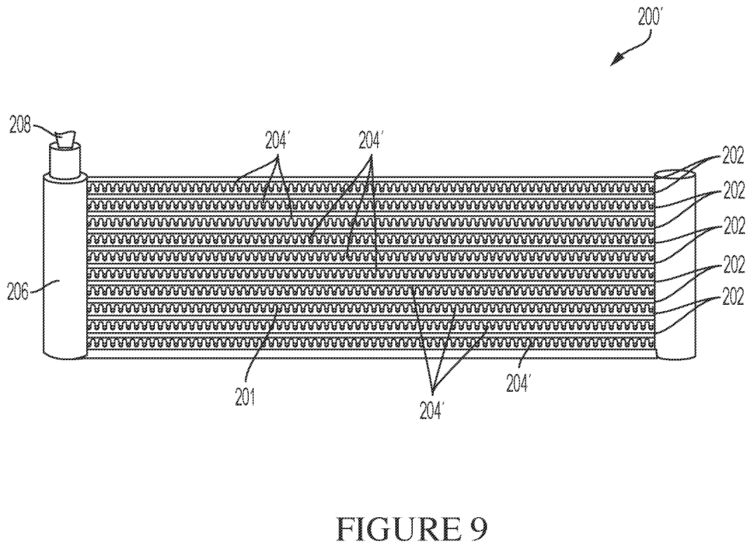

FIG. 9 shows a variation of the embodiment in FIG. 8 that is adapted for 3D cell culture. Specifically, FIG. 9 shows a cell culture vessel 200′ with a similar construction to that of vessel 200, but with a cell growth surface formed by the gas-permeable film 204′. The gas-permeable film 204′ has a 3D surface forming wells or microcavities for 3D cell culture therein.

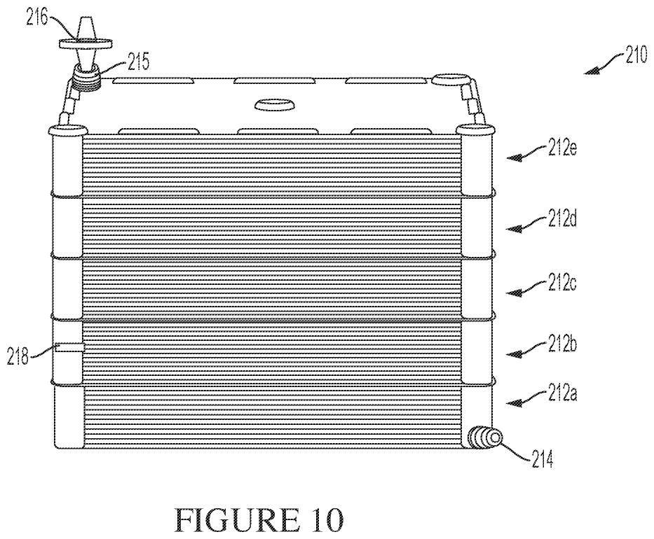

In some embodiments, multiples of the vessels 200 and 200′ in FIGS. 8 and 9 can be stacked or coupled together to form a larger cell culture vessel. In such a case, each of vessels 200 and 200′ act as individual modules of the larger vessel. FIG. 10 shows a front view of an example of such a vessel 210 that contains multiple cell culture modules 212a-e (similar to 200 or 200′). Though five modules 212a-e are shown in FIG. 10, it is contemplated that embodiments may have greater or fewer numbers of modules 212 in a vessel 210. An inlet 214 is provided in a lower module 212a, the inlet 214 being fluidly connected to the cell culture space within the lower module 212a. Each module 212a-e is coupled to the adjacent module(s) such that the cell culture spaces within all modules 212a-e are in fluid communication. Therefore, the entire cell culture space of vessel 210 can be filled with media via the inlet 214. A vent 216 is also provided at the outlet 215 of module 212e to allow gas to exit the cell culture space of the vessel 210. The vent 216 can be equipped with a filter, and is used during static cell culture for exhaust gases to pass therethrough. For example, the vent 216 permits air escape from the vessel 210 as the air is displaced by fluid filling the vessel 210, and also allows air entry into the vessel 210 when fluid is drained from the vessel 210 via inlet 214 during static culture. In perfusion culture, the outlet 215 can be coupled to tubing for carrying liquid out of the cell culture space of vessel 210. It is noted that the inlet 214 and outlet 215 are located in diagonally opposite corners of the vessel 210. That is, the inlet 214 is located at a lower front right corner (as viewed in FIG. 10) of the vessel 210, and the outlet 215 is located at an upper back left corner (as viewed in FIG. 10) of vessel 210. The relative positioning of the inlet 214 and outlet 215 can impact the manipulations required for filling and emptying processes. The diagonally opposite placement shown in FIG. 10 is one preferred embodiment, but other positions are contemplated as well.

Compared to the vessel in FIG. 1, which has the inlet (32) and outlet or vent (32′) on the same side of the vessel 10, the diagonally opposite inlet 214 and outlet 215 provide an advantage in that manipulation of the vessel 210 when being filled and emptied is greatly simplified and does not require the vessels to be spread out and individually manipulated. It also enables perfusion flow if desired by the user.

Optionally, one or more of the modules 212a-e can be provided with a sensor 218 for measuring a parameter of the cell culture, as discussed herein. The sensor 218 can be used to detect cell confluence or monitoring metabolites, for example.

In some embodiments, the vessel 210 in FIG. 10 can provide a cell culture surface of greater than 7,000 cm², greater than 14,000 cm², greater than 18,000 cm², greater than 36,000 cm², or greater than or equal to 50,000 cm². For example, in the embodiment of FIG. 10, the surface area is about 50,000 cm², which is greater than the surface area of 18,000 cm² provided by a commercially-available HYPERStack® unit. Viewed another way, the footprint occupied by the 50,000 cm² embodiment is smaller than the footprint occupied by the number of HYPERStack® units needed to match or exceed the surface area of 50,000 cm². In addition, the static head pressure required for operating the vessel 210 in FIG. 10 can be only about 0.5 psi, in some embodiments.

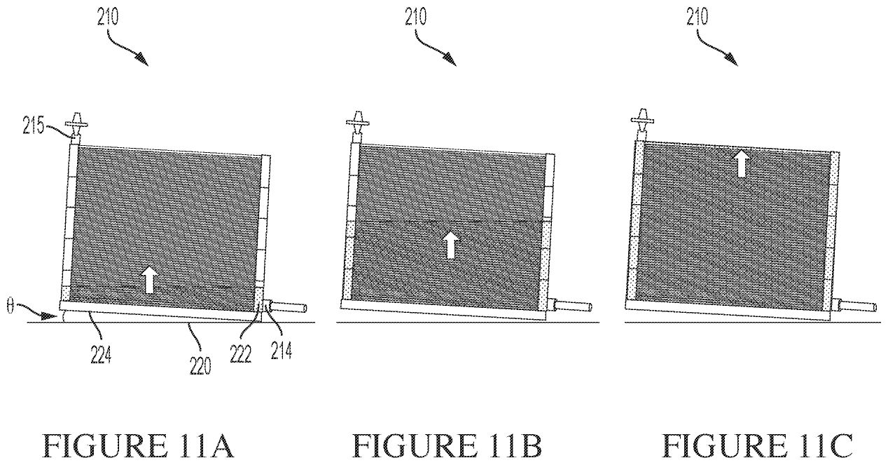

FIGS. 11A-C show three stages of a filling process for vessel 210. The vessel 210 is shown disposed on a horizontal or flat surface 220 (i.e., the surface 220 is parallel to the ground). During filling with media 222 through the inlet 214, the vessel 210 is tilted to a tilting angle θ, defined by the angle between the bottom surface 224 of the vessel 210 and the support surface 220. The filling angle θ allows the vessel 210 to be filled at a given filling pressure while minimizing bubble formation with the vessel 210, as air is exhausted through the outlet 215 as the level of the media 222 rises, as shown in FIGS. 11A-11C. Due to the horizontally opposite positioning of the inlet 214 and outlet 215 (see FIG. 10), a tilting angle θ of only 5° can be used, or an angle of from 1° to 10° can be used, or an angle from 5° to 20° can be used, or an angle from 10° to 45° can be used.

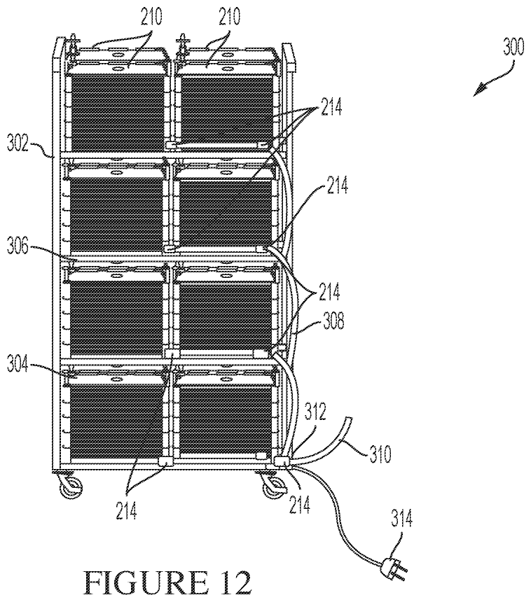

FIG. 12 shows an embodiment of a cell culture system 300 using a cabinet 302 to house multiple cell culture vessels 210. The form and structure of the vessels 210 corresponds to the vessel 210 of FIGS. 10 and 11, though the cabinet 302 can be adapted for use with other types of vessels, as well. The cabinet 302 includes an interior cavity 304 and may include one or more support surface 306 arranged to support the one or more cell culture vessels 210. As shown, multiple cell culture vessels 210 can be provided on each support surface 306, and multiple support surfaces 306 allow for a high-density cell culture system in a small footprint. The inlets 214 of the vessels 210 are manifolded together—in this example, by tubing 308 connecting the various inlets 214. Thus, all of the vessels 210 can be supplied with media via a main input line 310 connected to the vessels 210 via a ort 312 on the cabinet 302.

In operation, the vessels 210 are loaded into the interior cavity 304 of the cart 302 by a user. Next, the user can connect the various vessels 210 on a single support surface 306 via, e.g., tubing 308. These manifolded vessels 210 on each support surface 306 can be connected to the manifolded vessels on another of the support surfaces 306 via additional tubing 308. The main input line 310 is connected to either fresh medium or, if refeeding, a waste container (not pictured). Flow can be controlled using valves or clamps to limit filling to each shelf separately to avoid hydrostatic pressure build-up. The cart 302 further includes tilting capability to be used, for example, during filling or emptying of the vessels 210. The cart 302 can further include an electrical plug for powering systems within the cabinet 302. For example, the cabinet 302 can include electro-mechanical valves, electro-mechanical tilting mechanisms, or incubation systems for maintaining a heated environment for cell culture, as discussed herein.

In some embodiments, sensors are positioned before the vent filter 216 and are wirelessly connected to valves at each vessel inlet 214 to regulate the flow of medium to reach the appropriate fill level and prevent overfilling. A human/machine interface (HMI) on (or in communication with) the cart 302 can enable programming of sensors and valves to enable filling and emptying without manual clamp manipulation.

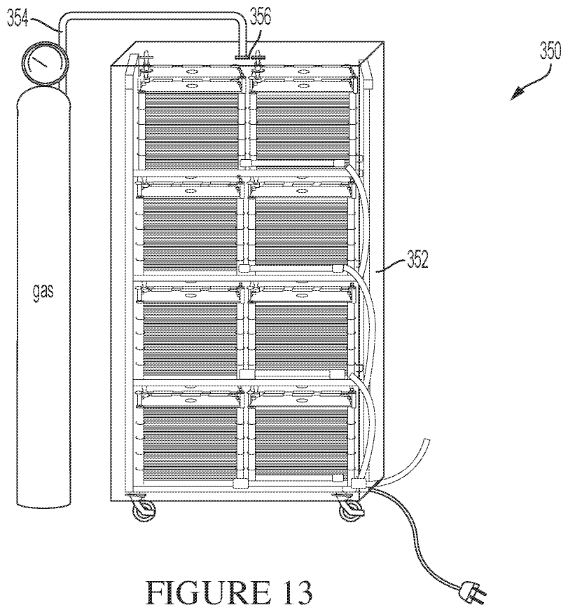

FIG. 13 shows another embodiment of the cell culture system 350 according to this disclosure. Similar to FIG. 12, in system 350, a cabinet 302 is provided to house multiple cell culture vessels 210. The components and structure of the cabinet 302 and vessels 210 that correspond to those discussed above will not be repeated with reference to FIG. 13. As shown, the cabinet 302 is surrounded by a gas impermeable enclosure 352 which is plumbed to accept tubing via a port 356 for controlling the gas in the interior cavity of the cabinet 302. The gas impermeable enclosure 352 may be soft-sided, or hard and may be built as part of the cart, or as a separate entity. The port 356 or additional ports through the gas-impermeable enclosure 352 are available for sensor connections or any other monitoring or control systems. The gas impermeable enclosure can act to retain heat generated by the cart, and distributes gas which may be humidified and heated.

According to some embodiments, multiple vessels can be coupled together via structural members or rails to form combined vessel units. These structural members can include rails along the sides and corners or edges of the vessels. The rails can have the effect of spacing the individual vessels while also provided structural support and coupling between the vessels. These rails can also protect the vessels during shipping or operating to prevent then from colliding with each other while allowing for a dense footprint.

Incubation may be required during cell culturing. In some embodiments, the cabinet 302 may incorporate a temperature control system that allows for incubation. In other embodiments, the cabinet 302, which can be mobile, can be moved into an incubator that houses the entire cabinet 302.

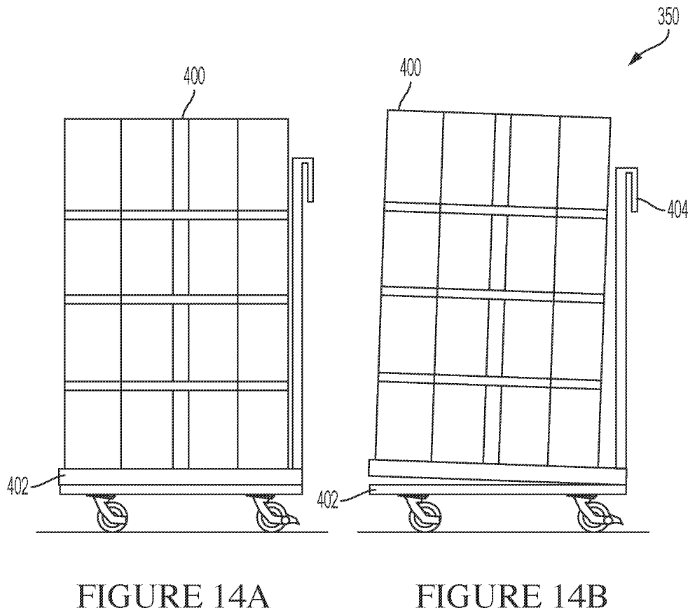

FIGS. 14A and 14B show another embodiment of the cell culture system. As described herein, the cell culture system can allow for tilting of the cell culture vessels while within a cabinet of the system. In some embodiments, this is accomplished by a tilting of the entire cabinet, as shown in FIG. 14B. For example, a cabinet 400 is provided on a support or cart 402 in an upright configuration, as shown in FIG. 14A. The cart 402 can also tilt the entire cabinet 400 to a tilted configuration when desired, as shown in FIG. 14B. For example, a tilt of only 5° can be sufficient for the systems disclosed herein. This tilting can be the only manipulation required during the culturing process, thus the cart 402 and cabinet can be of a simple construction to achieve this titling operation. The cart 402 also allows maintaining the vessels in a tight, high density format such that this can be done inside an incubator rather than requiring moving the vessels out of the incubator and across tables or carts for fluid exchange as is required with existing cell culture vessels. Alternatively, the incubator can contain a feature to cause a tilt instead of the cart. Optionally, the cart handle can be removed once the HYPERBioreactor is ready to incubate to save space.

Thus, embodiments of cell culture systems and related methods are disclosed. One skilled in the art will appreciate that the cell culture systems and methods described herein can be practiced with embodiments other than those disclosed. The disclosed embodiments are presented for purposes of illustration and not limitation.

### Illustrative Implementations

The following is a description of various aspects of implementations of the disclosed subject matter. Each aspect may include one or more of the various features, characteristics, or advantages of the disclosed subject matter. The implementations are intended to illustrate a few aspects of the disclosed subject matter and should not be considered a comprehensive or exhaustive description of all possible implementations.

Aspect 1 pertains to a cell culture system comprising: at least one multi-layered vessel configured for culturing cells, the multi-layered vessel comprising a cell culture space within the multi-layered vessel; and a cabinet comprising an interior cavity enclosed by one or more sidewalls, the cabinet being configured to house the multi-layered vessel within the interior cavity; wherein the cabinet is configured to change an orientation of the multi-layered vessel from an upright orientation to a tilted orientation.

Aspect 2 pertains to the cell culture system of Aspect 1, further comprising at least one sensor configured to sense a property within the cell culture space.

Aspect 3 pertains to the cell culture system of Aspect 2, wherein the sensor comprises at least one of a confluence monitor and an analyte monitor.

Aspect 4 pertains to the cell culture system of Aspect 2 or 3, wherein the sensor is integrated into the multi-layered vessel.

Aspect 5 pertains to the cell culture system of Aspect 2 or 3, wherein the sensor is attached to the cabinet and arranged to sense the property within the cell culture space when the multi-layered vessel is disposed in the cabinet.

Aspect 6 pertains to the cell culture system of any of Aspects 2-5, wherein the multi-layered vessel comprises at least one sensor window through which the sensor is configured to sense the property within the cell culture space.

Aspect 7 pertains to the cell culture system of any of Aspects 1-6, wherein the cabinet comprises multiple support surfaces each configured to support the at least one multi-layered vessel multi-layered vessel.

Aspect 8 pertains to the cell culture system of any of Aspects 1-7, wherein the at least one multi-layered vessel comprises a plurality of multi-layer cell culture modules.

Aspect 9 pertains to the cell culture system of Aspect 8, wherein at least some of the plurality of multi-layer cell culture modules vessels are coupled to one another.

Aspect 10 pertains to the cell culture system of any of Aspects 1-9, wherein the multi-layered vessel comprises an inlet and an outlet, the inlet being configured to supply liquid media to the cell culture space and the outlet is configured for passing liquid or gas into or out of the cell culture space.

Aspect 11 pertains to the cell culture system of Aspect 10, wherein the inlet is disposed in a lower portion of the multi-layered vessel.

Aspect 12 pertains to the cell culture system of Aspect 10 or 11, wherein the outlet is disposed in an upper portion of the multi-layered vessel.

Aspect 13 pertains to the cell culture system of Aspect 12, wherein the outlet is disposed diagonally opposite the multi-layered vessel from the inlet.

Aspect 14 pertains to the cell culture system of any of Aspects 10-13, wherein the outlet comprises a vent port configured to permit gas to escape from or enter into the cell culture space.

Aspect 15 pertains to the cell culture system of any of Aspects 10-14, wherein the outlet comprises a filter.

Aspect 16 pertains to the cell culture system of any of Aspects 1-15, wherein the multi-layered vessel comprises a cell culture surface area of at least 18,000 cm².

Aspect 17 pertains to the cell culture system of Aspect 16, wherein the cell culture surface area is about 50,000 cm².

Aspect 18 pertains to the cell culture system of any of Aspects 1-17, wherein, in the tilted orientation, a bottom of the multi-layered vessel is at about a 5° angle relative to horizontal.

Aspect 19 pertains to the cell culture system of any of Aspects 1-18, wherein the tilted orientation comprises a rotation of about 5° relative to the upright orientation.

Aspect 20 pertains to the cell culture system of any of Aspects 7-19, wherein multiple multi-layered vessels are disposed on each of the multiple support surfaces.

Aspect 21 pertains to the cell culture system of Aspect 20, wherein the inlets of the multiple multi-layered vessels disposed on one of the multiple support surfaces are manifolded together.

Aspect 22 pertains to the cell culture system of Aspect 20 or 21, wherein the inlets of the multiple multi-layered vessels disposed on the multiple support surfaces are manifolded together.

Aspect 23 pertains to the cell culture system of any of Aspects 1-22, wherein the cabinet comprises a main inlet fluidly connected to the cell culture space of the at least one multi-layered vessel and configured to supply liquid media to the cell culture space.

Aspect 24 pertains to the cell culture system of Aspect 23, wherein the main inlet is fluidly connected to multiple multi-layered vessels.

Aspect 25 pertains to the cell culture system of Aspects 1-24, wherein the cabinet comprises a gas port configured to supply gas to the interior cavity.

Aspect 26 pertains to the cell culture system of Aspect 25, further comprising a gas supply fluidly connected to the gas port.

Aspect 27 pertains to the cell culture system of Aspects 1-26, further comprises a temperature control system configured to control a temperature of the interior cavity.

Aspect 28 pertains to the cell culture system of Aspect 27, wherein the temperature control system comprises at least one of a heat source and a cooling system.

Aspect 29 pertains to the cell culture system of Aspects 1-28, wherein the cabinet is configured to change the orientation of the multi-layered vessel by changing an orientation of the cabinet.

Aspect 30 pertains to the cell culture system of Aspect 29, wherein the orientation of the multi-layered vessel is fixed with respect to the cabinet.

Aspect 31 pertains to the cell culture system of Aspects 1-30, wherein the one or more sidewalls comprise an opening to the interior cavity, the opening sized to allow insertion or removal of the multi-layered vessel.

Aspect 32 pertains to the cell culture system of Aspect 31, wherein the cabinet comprises a door covering the opening, the door being configured to seal the interior cavity when the multi-layered vessel is disposed in the interior cavity.

Aspect 33 pertains to the cell culture system of Aspects 1-32, wherein the cabinet comprises a gas-impermeable enclosure within the interior cavity, the gas-impermeable enclosure being configured to enclosure the at least one multi-layered vessel.

Aspect 34 pertains to the cell culture system of Aspects 25-33, wherein the gas port is coupled to an opening in the gas-impermeable enclosure.

Aspect 35 pertains to the cell culture system of Aspects 1-34, further comprises an incubation enclosure configured to house the cabinet.

Aspect 36 pertains to the cell culture system of Aspect 35, wherein the incubation enclosure comprises one or more ports configured for at least one of supplying liquid media to the multi-layered vessel and carrying signals from sensors of the cell culture system to an exterior of the incubation enclosure.

Aspect 37 pertains to the cell culture system of any of Aspects 1-28, wherein the orientation of the multi-layered vessel is variable with respect to an orientation of the cabinet.

Aspect 38 pertains to the cell culture system of any of Aspects 1-37, wherein the multi-layered vessel comprises a gas-permeable substrate separating the cell culture space from the interior cavity.

Aspect 39 pertains to the cell culture system of any of Aspects 1-36, wherein the multi-layered vessel comprises at least one of a 2D adherent cell culture film and a 3D microcavity film.

1. A cell culture system comprising:
at least one multi-layered vessel configured for culturing cells, the multi-layered vessel comprising a cell culture space within the multi-layered vessel; and
a cabinet comprising an interior cavity enclosed by one or more sidewalls, the cabinet being configured to house the multi-layered vessel within the interior cavity;
wherein the cabinet is configured to change an orientation of the multi-layered vessel from an upright orientation to a tilted orientation.

2. The cell culture system of claim 1, further comprising at least one sensor configured to sense a property within the cell culture space.

3. The cell culture system of claim 2, wherein the sensor comprises at least one of a confluence monitor and an analyte monitor.

4. The cell culture system of claim 2, wherein the sensor is integrated into the multi-layered vessel.

5. The cell culture system of claim 2, wherein the sensor is attached to the cabinet and arranged to sense the property within the cell culture space when the multi-layered vessel is disposed in the cabinet.

6. The cell culture system of claim 2, wherein the multi-layered vessel comprises at least one sensor window through which the sensor is configured to sense the property within the cell culture space.

7. The cell culture system of claim 1, wherein the cabinet comprises multiple support surfaces each configured to support the at least one multi-layered vessel multi-layered vessel.

8. The cell culture system of claim 1, wherein the at least one multi-layered vessel comprises a plurality of multi-layer cell culture modules.

9. The cell culture system of claim 8, wherein at least some of the plurality of multi-layer cell culture modules vessels are coupled to one another.

10. The cell culture system of claim 9, wherein the multi-layered vessel comprises an inlet and an outlet, the inlet being configured to supply liquid media to the cell culture space and the outlet is configured for passing liquid or gas into or out of the cell culture space.

11. The cell culture system of claim 10, wherein the inlet is disposed in a lower portion of the multi-layered vessel.

12. The cell culture system of claim 11, wherein the outlet is disposed in an upper portion of the multi-layered vessel.

13. The cell culture system of claim 12, wherein the outlet is disposed diagonally opposite the multi-layered vessel from the inlet.

14. The cell culture system of claim 10, wherein the outlet comprises a vent port configured to permit gas to escape from or enter into the cell culture space.

15. The cell culture system of claim 14, wherein the outlet comprises a filter.

16. The cell culture system of claim 1, wherein the multi-layered vessel comprises a cell culture surface area of at least 18,000 cm².

17. The cell culture system of claim 16, wherein the cell culture surface area is about 50,000 cm².

18. The cell culture system of claim 1, wherein, in the tilted orientation, a bottom of the multi-layered vessel is at about a 5° angle relative to horizontal.

19. The cell culture system of claim 1, wherein the tilted orientation comprises a rotation of about 5° relative to the upright orientation.

20. The cell culture system of claim 7, wherein multiple multi-layered vessels are disposed on each of the multiple support surfaces.

21. The cell culture system of claim 20, wherein the inlets of the multiple multi-layered vessels disposed on one of the multiple support surfaces are manifolded together.

22. (canceled)

23. The cell culture system of claim 1, wherein the cabinet comprises a main inlet fluidly connected to the cell culture space of the at least one multi-layered vessel and configured to supply liquid media to the cell culture space.

24. The cell culture system of claim 23, wherein the main inlet is fluidly connected to multiple multi-layered vessels.

25. The cell culture system of claim 23, wherein the cabinet comprises a gas port configured to supply gas to the interior cavity.

26. The cell culture system of claim 25, further comprising a gas supply fluidly connected to the gas port.

27. The cell culture system of claim 1, further comprises a temperature control system configured to control a temperature of the interior cavity.

28. The cell culture system of claim 27, wherein the temperature control system comprises at least one of a heat source and a cooling system.

29. The cell culture system of claim 1, wherein the cabinet is configured to change the orientation of the multi-layered vessel by changing an orientation of the cabinet.

30. The cell culture system of claim 29, wherein the orientation of the multi-layered vessel is fixed with respect to the cabinet.

31. The cell culture system of claim 1, wherein the one or more sidewalls comprise an opening to the interior cavity, the opening sized to allow insertion or removal of the multi-layered vessel.

32. The cell culture system of claim 31, wherein the cabinet comprises a door covering the opening, the door being configured to seal the interior cavity when the multi-layered vessel is disposed in the interior cavity.

33. The cell culture system of claim 1, wherein the cabinet comprises a gas-impermeable enclosure within the interior cavity, the gas-impermeable enclosure being configured to enclosure the at least one multi-layered vessel.

34. The cell culture system of claim 33, wherein the gas port is coupled to an opening in the gas-impermeable enclosure.

35. The cell culture system of claim 1, further comprises an incubation enclosure configured to house the cabinet.

36. The cell culture system of claim 35, wherein the incubation enclosure comprises one or more ports configured for at least one of supplying liquid media to the multi-layered vessel and carrying signals from sensors of the cell culture system to an exterior of the incubation enclosure.

37. The cell culture system of claim 1, wherein the orientation of the multi-layered vessel is variable with respect to an orientation of the cabinet.

38. The cell culture system of claim 1, wherein the multi-layered vessel comprises a gas-permeable substrate separating the cell culture space from the interior cavity.

39. The cell culture system of claim 1, wherein the multi-layered vessel comprises at least one of a 2D adherent cell culture film and a 3D microcavity film.
